(19) United States
(12) Patent Application Publication
(10) Pub. No.: US 2004/0175710 A1
(43) Pub. Date: Sep. 9, 2004

(54) Method for in situ, on-chip chemical synthesis

(76) Inventor: Robert C. Haushalter, Los Gatos, CA (US)

Correspondence Address: DUANE MORRIS LLP, 100 COLLEGE ROAD WEST, SUITE 100, PRINCETON, NJ, 08540-6604, US

(21) Appl. No.: 10/477,085

(22) PCT Filed: May 22, 2002
(86) PCT No.: PCT/US02/16403

Related U.S. Application Data
(60) Provisional application No. 60/292,788, filed on May 22, 2001.

(51) Int. Cl. C12Q 1/68; B05D 3/00; C12M 1/34

(52) U.S. Cl. 435/6; 435/287.2; 427/2.11

(57) ABSTRACT

Apparatus and methods for the synthesis of arrays of molecules bound to a substrate, and in particular oligomeric molecules such as DNA oligonucleotides and peptides. The methods of the invention comprise providing a substrate having first and second surfaces and a plurality of isolated porous regions extending through the substrate and communicating with the first and second surfaces, contacting selected ones of the porous regions with a reagent, allowing the reagent to bind to or otherwise interact with the selected porous regions, and withdrawing unreacted first reagent from the substrate through the selected porous regions by introducing a pressure differential across the substrate. The events may be repeated as required to form oligomeric molecules.

### CROSS-REFERENCE

This application claims the benefit of U.S. Provisional Patent Application No. 60/292,788 filed May 22, 2001.

### BACKGROUND OF THE INVENTION

In the fields of molecular biology and biochemistry, biopolymers such as nucleic acids and proteins from organisms are identified and fractionated in order to search for useful genes and to diagnose diseases. A hybridization reaction is frequently used as a pretreatment for such processes, where a target molecule in a sample is hybridized with a nucleic acid having a known sequence. For this purpose, microarrays or “biochips” or DNA chips are used with probes such as DNAs, RNAs, peptides or proteins with known sequences immobilized at predetermined positions.

The use of DNA arrays for numerous applications has expanded rapidly in recent years. Detection of polymorphisms due to gene mutations, and particularly single base (codon) mutations, is not only effective for diagnosis of cancer and other diseases resulting from mutations, but also necessary for indication of drug responsiveness and side-effects, and can be helpful for the analysis of the causative genes of multiple factor diseases and for predictive medicine. The “Gene Chip” by Affymetrix, which is a DNA chip containing immobilized short DNA chains, usually comprises over 10,000 oligo DNA fragments (DNA probes) mounted on an approximately 1 cm square silicon or glass plate using a photolithographic technique.

Many of the potential advantages of the biochips derive from the benefits of mass production resulting in a large number of experiments performed per unit cost in a small physical area. Microarrays comprising an ordered array of biological molecules (e.g., peptides, oligonucleotides) on a solid surface are known. See, for example, U.S. Pat. Nos. 5,445,934; 5,510,270; 5,605,662; 5,632,957; 5,744,101; 5,807,522 5,929,208 and PCT publication WO 99/19510.

Currently, there are two primary methods for producing DNA microarrays. One is a method in which oligomers such as oligonucleotides are synthesized directly on a support, and the other is a method in which pre-prepared samples such as cDNAs are spotted onto a support. Microarray production methods wherein oligomers are synthesized directly on a microarray support utilize consecutive micro-volume fluid reactions and washing steps to produce oligomers, such as small DNA oligonucleotides of <100 bases.

Presently, there are two major technologies for the preparation of custom oligonucleotide arrays wherein nucleic acid oligomers are synthesized in-situ on the array substrate. One method builds the oligonucleotide chains on the chip using photolabile blocking groups to place the correct base at a desired location on the array, and the other dispenses the DNA synthesis chemicals directly onto a chip into predefined surface features that behave somewhat like small vessels. Currently, DNA synthesis for genomics research involves one of the following; DNA arrays built up via photolabile linker chemistry, DNA arrays prepared on glass substrates with oligomers of less than 100 bases or by “bulk” synthesis of DNA on controlled pore glass (CPG) using a laboratory DNA synthesis machine usually in a 96 well format. The Affymetrix “Genechip” approach to DNA synthesis requires large pieces of hardware and requires a significant amount of time to complete a DNA synthesis. Traditional mechanical laboratory DNA synthesis machines currently make far more DNA than is needed for most experiments. But the DNA is too expensive to make to discard and therefore the synthesized DNA must be stored frozen, archived and tracked at considerable expense.

Recently, flow-through chips have been developed for assays using DNA probe arrays. The flow through regions of these chips are fabricated from drawing down glass fiber bundles, electrochemical oxidizing silicon or anodically etching aluminum. There are two basic types of chip which possess flow-through, or at least porous, regions. The porous chips, of which gel-pads or porous organic polymers are examples, have increased surface areas capable of binding more of the materials of interest, but the non-monodisperse pore size distributions can often lead to decreased diffusion rates into and out of the matrix. Incomplete reactions and the inability to completely remove reagent from the reaction zone are extremely detrimental to oligonucleotide synthesis, where nearly quantitative yields are absolutely necessary.

Many of these technologies, while breakthroughs in genomic research, are beyond the economic reach of most laboratories. Both science and society could greatly benefit from the development of a robust, inexpensive and rapid method for the synthesis of DNA oligonucleotide arrays. Thus there is a need for methods for producing DNA chips which would decrease inter-well cross-contamination during synthesis, enable easy production of custom arrays, increase array density, prepare small and economically sensible amounts of material, reduce the cost of DNA and improve efficiency of oligomer coupling chemistry. The present invention satisfies these needs, as well as others, and overcomes deficiencies found in the background art.

Relevant Literature:

Schena, M; Shalon, D; Davis, R W; Brown, P O. Science, 1995, 270:467-70; Chee, M; Yang, R; Hubbell, E; Berno, A; Huang, X C; Stern, D; Winkler, J; Lockhart, D J; Morris, M S; Fodor, S P. Science, 1996, 274:6104; “The Chipping Forecast” Nature Genetics, 1999, 21, Supplement Issue; Hacia, J G; Brody, L C; Chee, M S; Fodor, S P; Collins, F S., Nature Genetics, 1996, 14:441-7; Beaucage, S L; Caruthers, M H. Tetrahedron Letters, 1981, 22:1859-62; McBride, L J; Caruthers, M H. Tetrahedron Letters, 1983, 24:24548; U.S. Pat. No. 5,843,767; U.S. Pat. No. 5,175,209; Steel, A., et al., Chapter 5, Microarray Biochip Technology, Schena, M., Ed., Eaton Publishing, Natick Mass. 2000 and references therein.

### SUMMARY OF THE INVENTION

The invention provides apparatus and methods for the synthesis of arrays of molecules bound to a substrate, and in particular oligomeric molecules such as DNA oligonucleotides and peptides. The invention integrates a micromachined silicon chip design, which captures several of the key advantages of a laboratory DNA synthesis machine in a compact chip-based format, with fluid handling and dispensing systems to rapidly prepare DNA oligomers of arbitrary or selectable sequence.

The methods of the invention comprise, in one embodiment, providing a substrate having first and second surfaces and a plurality of isolated porous regions extending through the substrate and communicating with the first and second surfaces, contacting selected ones of the porous regions with a first reagent, allowing the first reagent to bind to or otherwise interact with the selected porous regions, and withdrawing unreacted first reagent from the substrate through the selected porous regions by introducing a pressure differential across the substrate. The number of porous regions in the substrate may, in certain embodiments, be greater than around 96 porous regions per substrate, and in specific embodiments, may be greater than around 384 porous regions per substrate.

The methods may further comprise contacting the selected ones of the porous regions with a wash solution, and withdrawing the wash solution through the selected porous regions by introducing a pressure differential across the substrate. In certain embodiments, the methods may additionally comprise contacting the selected ones of the porous regions with a second reagent, allowing the second reagent to bind to or react with the first reagent at the selected porous regions, and withdrawing unreacted second reagent from the substrate through the selected porous regions by introducing a pressure differential across the substrate. The aforementioned events may be repeated with third, fourth or additional reagents as required to form molecules of interest in association with the porous regions.

The methods of the invention may, in other embodiments, comprise providing a substrate having first and second surfaces, and a plurality of isolated porous regions extending through the substrate and communicating with the first and second surfaces, contacting selected ones of the porous regions with a first reagent including a first monomer allowing the first monomer to bind to the selected porous regions, withdrawing excess first reagent from the substrate through the selected porous regions, contacting selected ones of the porous regions with a second reagent including a second monomer, allowing the second monomer to couple to the first monomer, withdrawing excess second reagent from the substrate through the selected porous regions, and repeating the events with appropriate monomers and reagents until the desired oligomers are formed on the porous regions of the substrate. In certain embodiments, the methods may further comprise contacting the porous regions with a surface modifier capable of allowing the first monomer to bind to the porous regions. By way of example, and not of limitation, the oligomeric molecules may comprise nucleic acids such as DNA, peptides, or other oligomeric molecules.

The withdrawing of excess reagent from the substrate through the selected porous regions may comprise introducing a pressure differential across the substrate. This may be achieved via the application of increased or decreased pressure on the appropriate side of the substrate, osmotic or electro-osmotic pressure, the use of electric fields, capillary action, gravity, centrifugal force or other techniques.

The invention also provides methods for analyzing DNA molecules using an array formed by the above method, comprising applying a labeled target DNA molecule to the array of nucleic acids, allowing the labeled target DNA molecule to hybridize with the nucleic acids, washing the array to remove unhybridized labeled target DNA molecule therefrom, and detecting the labeled target DNA molecule on the array. The labeled target DNA may comprise a fluorescent label, a magnetic label, or other form of label, and may be detected by fluorescence spectroscopy, magnetic detection, or other detection technique.

The apparatus of the invention comprise a substrate having a plurality of isolated mesh or porous regions extending therethrough, and oligomeric organic molecules attached at selected ones of the porous regions that are grown or synthesized in a stepwise manner on or adjacent to the porous regions. The apparatus may further comprise a base including a plurality of channels that is joined to the substrate, with the plurality of channels communicating with corresponding ones of the plurality of porous regions. The apparatus may further comprise a plurality of gasket regions corresponding ones of the plurality of channels and configured to sealingly engage the substrate and base. The gasket may be a free standing piece of the apparatus or may comprise a material that is directly coated to the substrate to form the gasketing layer.

The apparatus of the invention provides a small DNA synthesis machine, which may be on the order of −200 cm³ volume, capable of synthesizing arrays of at least about 200 oligonucleotides, with 50-70 nucleotides per oligomer, in a few hours at a cost substantially less than the current large synthesis machines. The apparatus may comprise a flow-through silicon chip that is produced by a variety of microfabrication processes to prepare an array of textured, flow-through silicon micromesh grids, the oxidized SiO₂ surface of which forms the substrate on which the covalently attached oligonucleotides are grown. The reagents required for the DNA synthesis using the methods of the invention are minimal compared to conventional DNA synthesizers, utilizing only between 0.2 and 5 mL per array for a 1536 well array. It is clear that higher density arrays contain more and smaller samples and therefore the higher density arrays will use less valuable and expensive synthesis chemicals. The apparatus of the invention may also be configured to contain the spent reagents within the device for ease of disposal of hazardous chemicals and to immobilize the chemicals eliminating any cross contamination of the samples by used reagents.

The methods of the invention may use a liquid dispenser together with the apparatus for a staged reagent removal system to carefully control oligomeric synthesis reaction times. The apparatus is capable of forcing the equilibrium of reactions to completion by flowing reagents through the grid regions of the apparatus and to thoroughly wash each sample by placing the oligomer synthesis reagents on the mesh/grid and then drawing them through the mesh/hole regions of each chip, thereby simulating the flow-through geometry seen with a large DNA synthesizer.

The invention provides methods for hybridizing nucleic acids using nucleic acid arrays prepared in accordance with the invention comprising applying a target nucleic acid molecule to array of nucleic acids, allowing the labeled target nucleic acid molecule to hybridize with the nucleic acids on the array, and washing the array to remove unhybridized labeled target nucleic acid molecule therefrom. The target nucleic acid may be labeled, and the method may further comprise detecting the labeled target nucleic acid on the array.

For DNA oligomer synthesis, the apparatus in many embodiments utilizes a software controlled liquid dispensing device which translates relative to the array to deliver the appropriate chemical to the selected chip address at the correct time and sequence, to deliver phosphoramidite reagents, or other DNA synthesis chemicals, to the individual porous regions or grid elements or porous regions of the apparatus, which are configured to eliminate any well-to-well cross contamination. The liquid can be delivered to the substrate by many of the small volume dispensing devices well known to those skilled in the art such as piezoelectric, inkjets, syringe-solenoid, electrostatic and thermopneumatic dispensers. After liquid dispensing of the reagents, a suitable incubation reaction time is allowed for reaction of the reagents at the grid regions, after which a pressure differential is introduced across the substrate and excess or unreacted reagents are drawn through the grid for disposal. Wash steps are carried out in a similar manner after removal of the phosphoramidite or other reagents. The dispensing, incubation, removal and washing acts are repeated to grow the oligonucleotide chain on the grid mesh or porous regions of the apparatus.

An integrated DNA synthesis system including the apparatus of the invention may comprise; a silicon microgrid flow through chip, a flow through support wafer, liquid dispenser capable of bringing the DNA synthesis chemicals to the appropriate chip address, an x-y motion control system, a vacuum or pressure source, power connections for x-y motion control, software for motion/dispense control and vacuum/dispense synchronization, and a pressurized inert gas source and regulator.

An advantage of the flow through oligomeric synthesis chip of the invention is the reduction of assay time due to a more efficient mass transport, higher array densities due to improved wetting, smaller required sample and reagent volumes and improved responsiveness and dynamic range as well as increased surface area.

Another advantage of the invention is that the flow through chip design helps to force chemical reactions further toward completion by supplying flowing fresh reagents.

Another advantage of the invention is that, by removing the reaction products, the apparatus eliminates the use of large volumes of solvent for the washing/flooding of the substrate between reaction steps.

Yet another advantage of the invention is that the apparatus provides well isolated synthesis areas, thereby eliminating chemical cross contamination from adjacent grids or porous regions.

Still another advantage of the of the invention is to produce a chip containing a high-density DNA array produced on a removable silicon chip in a size convenient for handling, analysis and archiving, that also provides oligomers in a form readily cleavable from the substrate and available for the subsequent amplification and coupling chemistries.

Another advantage of the invention is the ability to dose reagents multiple times if desired, to improve the reaction kinetics and degree of reaction completion, respectively vastly reducing reagent/waste disposal costs.

An object of the invention is to provide a small, inexpensive modular DNA synthesis system capable of rapidly preparing high density oligonucleotide arrays which is cost effective.

Another object of the methods of the invention is to provide the fabrication of custom DNA oligomer arrays or gene chips faster and less expensive than existing methods.

Another object of the invention is to allow the production of large oligonucleotides of 100,000 bases or more by producing smaller oligomers on a flow through substrate and then cleaving the oligomers from the silicon substrate, amplifying them by PCR or other techniques, and assembling the resulting oligomers into the large oligomer of interest.

These and other objects, advantages, and features of the invention will become apparent to those persons skilled in the art upon reading the following description of the invention, which is for illustrative purposes only.

### BRIEF DESCRIPTION OF THE DRAWINGS

FIG. 1 is a perspective exploded view of one embodiment of a flow through microarray substrate apparatus in accordance to the invention.

FIG. 2 is a schematic view in cross section of the apparatus of FIG. 1.

FIG. 3A-3C are schematic illustrations of use of the apparatus of FIG. 1 and FIG. 2 to synthesize DNA oligomers in accordance with the invention.

FIG. 4A is a schematic side elevation view in cross-section of a grid element of the microarray apparatus of the invention.

FIG. 4B is a schematic top plan view of the grid element of FIG. 4A

FIG. 4C and FIG. 4D are electron microscopy images of a micromachined grid element usable as a porous region of the microarray apparatus of the invention.

FIG. 5 is a pair of electron microscopy images showing a detail of the etched micromesh grids on a silicon wafer of a microarray apparatus of the invention.

FIGS. 6A-D are schematic illustrations of another embodiment of an apparatus for use in step wise or in-situ synthesis of oligomers on a microarray. FIGS. 5A, 5B and 5C are side elevation views of the apparatus shown in cross-section, and FIG. 5D is a top plan view of a portion of the apparatus.

FIGS. 7A and 7B are illustrations of surface modification schemes for providing a linker for attaching nucleotides to the grid surface of a microarray in accordance with the invention.

### DEFINITIONS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an oligomer” includes a plurality of such oligomers and reference to “the probe” includes reference to one or more probes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

By “array of regions on a solid support” we include the meaning of a linear or two-dimensional array of preferably discrete regions, each having a finite area or volume, formed on the surface of a solid support. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g., plastic, polymer, perspex, silicon, amongst others), a plurality of polymeric pins, or a plurality of microtitre wells, or any other surface suitable for immobilizing proteins and/or conducting an immunoassay. The binding processes are well-known in the art and generally consist of cross-linking covalently binding or physically adsorbing the protein molecule to the solid support.

The term “array” or “microarray” used herein refers to a two-dimensional arrangement of features such as an arrangement of reservoirs (e.g., wells in a well plate) or an arrangement of different materials including ionic, metallic or covalent crystalline, including molecular crystalline, composite or ceramic, glassine, amorphous, fluidic or molecular materials on a substrate surface (as in an oligonucleotide or peptidic array). Different materials in the context of molecular materials includes chemical isomers, including constitutional, geometric and stereoisomers, and in the context of polymeric molecules constitutional isomers having different monomer sequences. Arrays are generally comprised of regular, ordered features, as in, for example, a rectilinear grid, parallel stripes, spirals, and the like, but non-ordered arrays may be advantageously used as well. The arrays or patterns formed using the devices and methods of the invention generally have no optical significance to the unaided human eye. For example, the invention does not involve ink printing on paper or other substrates in order to form letters, numbers, bar codes, figures, or other inscriptions that have optical significance to the unaided human eye. In addition, arrays and patterns formed by the deposition of ejected droplets on a porous surface as provided herein are preferably substantially invisible to the unaided human eye.

It will be appreciated that, as used herein, the terms “nucleoside” and “nucleotide” refer to nucleosides and nucleotides containing not only the conventional purine and pyrimidine bases, i.e., adenine (A), thymine (T), cytosine (C), guanine (G) and uracil (U), but also protected forms thereof, e.g., wherein the base is protected with a protecting group such as acetyl, difluoroacetyl, trifluoroacetyl, isobutyryl or benzoyl, and purine and pyrimidine analogs. Suitable analogs will be known to those skilled in the art and are described in the pertinent texts and literature. Common analogs include, but are not limited to, 1-methyladenine, 2-methyladenine, N.sup.6-methyladenine, N.sup.6-isopentyladenine, 2-methylthio-N.sup.6-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylanminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “oligonucleotide” shall be generic to polydeoxynucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones (for example PNAs), providing that the polymers contain nucleobases in a configuration that allows for base pairing and base stacking, such as is found in DNA and RNA. Thus, these terms include known types of oligonucleotide modifications, for example, substitution of one or more of the naturally occurring nucleotides with an analog, inter-nucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.). There is no intended distinction in length between the term “polynucleotide” and “oligonucleotide,” and these terms will be used interchangeably. These terms refer only to the primary structure of the molecule. As used herein the symbols for nucleotides and polynucleotides are according to the IUPAC-IUB Commission of Biochemical Nomenclature recommendations (Biochemistry 9:4022, 1970).

The term “surface modification” as used herein refers to the chemical and/or physical alteration of a surface by an additive or subtractive process to change one or more chemical and/or physical properties of a substrate surface or a selected site or region of a substrate surface. For example, surface modification may involve (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

The term “attached,” as in, for example, a substrate surface having a moiety “attached” thereto, includes covalent binding, adsorption, and physical immobilization. The terms “binding” and “bound” are identical in meaning to the term “attached.”

The terms “peptide,” “peptidyl” and “peptidic” as used throughout the specification and claims are intended to include any structure comprised of two or more amino acids. For the most part, the peptides in the present arrays comprise about 5 to 10,000 amino acids, preferably about 5 to 1000 amino acids. The amino acids forming all or a part of a peptide may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Any of the amino acids in the peptidic molecules forming the present arrays may be replaced by a non-conventional amino acid. In general, conservative replacements are preferred. Conservative replacements substitute the original amino acid with a non-conventional amino acid that resembles the original in one or more of its characteristic properties (e.g., charge, hydrophobicity, stearic bulk; for example, one may replace Val with Nval). The term “non-conventional amino acid” refers to amino acids other than conventional amino acids, and include, for example, isomers and modifications of the conventional amino acids (e.g., D-amino acids), non-protein amino acids, post-translationally modified amino acids, enzymatically modified amino acids, constructs or structures designed to mimic amino acids (e.g., .alpha.,.alpha.-disubstituted amino acids, N-alkyl amino acids, lactic acid, .beta.-alanine, naphthylalanine, 3-pyridylalanine, 4-hydroxyproline, O-phosphoserine, N-acetylserine, N-formylmethionine, 3-methylhistidine, 5-hydroxylysine, and nor-leucine), and peptides having the naturally occurring amide —CONH— linkage replaced at one or more sites within the peptide backbone with a non-conventional linkage such as N-substituted amide, ester, thioamide, retropeptide (—NHCO—), retrothioamide (—NHCS—), sulfonamido (—SO.sub.2NH—), and/or peptoid (N-substituted glycine) linkages. Accordingly, the peptidic molecules of the array include pseudopeptides and peptidomimetics. The peptides of this invention can be (a) naturally occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides.

The term “oligomer” is meant to encompass any polynucleotide or polypeptide or other chemical compound with repeating moieties such as nucleotides, amino acids, carbohydrates and the like.

### DETAILED DESCRIPTION OF THE INVENTION

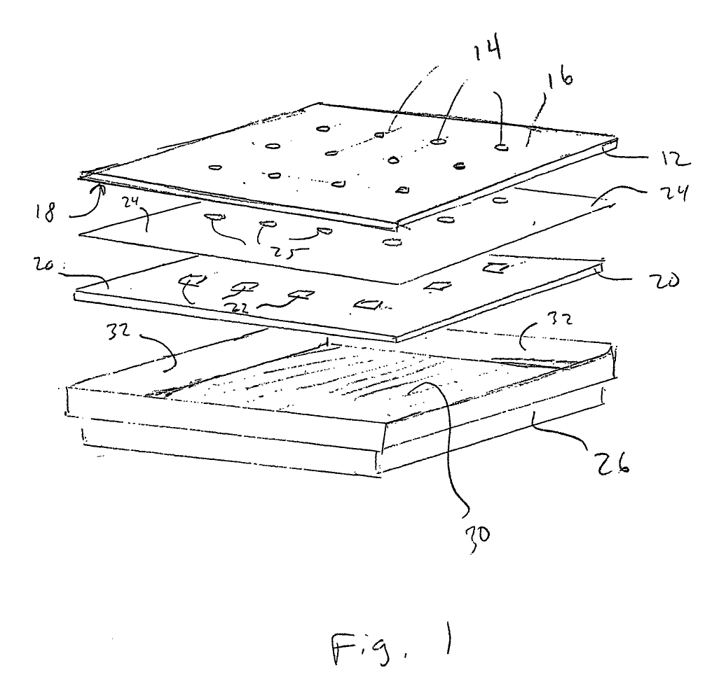

With the above in mind, reference is made more specifically to the drawings in which, for illustrative purposes, show the present invention embodied in systems and methods in FIG. 1 through FIG. 7. It will be appreciated that the apparatus may vary as to configuration and as to details of the parts, and that the methods may vary as to detail and the order of the events or acts, without departing from the basic concepts as disclosed herein. The invention is described primarily in terms of use with DNA oligomers. It should be understood, however, that the invention may be used with a variety of different types of molecules, including RNA or other nucleic acids, peptides, proteins, or other molecules of interest.

Overview of the Invention

The present invention provides apparatus and methods useful in the preparation and the synthesis of microarrays of molecules of interest, including nucleic acids, polypeptides, proteins and combinations thereof. The apparatus comprises an array of grid regions micromachined directly onto and through a substrate element whereby oligomers can be synthesized on the discrete mesh grids. The apparatus allows the chemical reagents involved in oligomer synthesis to incubate on the grid mesh of the top surface of the substrate element and after an appropriate reaction time, the used reagents are removed from the mesh grid regions by producing a pressure change on the bottom of the substrate element, drawing the reagent through the grid mesh. In preferred embodiments, the apparatus and methods of the invention are used to rapidly synthesize a high-density DNA oligomer array using standard phosphoramidite chemistry.

The microarray apparatus of the invention provides for the synthesis of DNA oligomers for microarrays as well as for use in PCR or other amplification techniques and also for assembling larger DNA oligomers by cleaving the synthesized DNA from the DNA synthesis apparatus of the invention.

The microarray apparatus of the invention may be used for nucleic acid hybridization studies such as gene expression analysis, genotyping, heteroduplex analysis, nucleic acid sequencing determinations based on hybridization, synthesis of DNA, RNA, peptides, proteins or other oligomeric or non-oligomeric molecules, combinatorial libraries for evaluation of candidate drugs.

DNA and RNA synthesized in accordance with the invention may be used in any application including, by way of example, probes for hybridization methods such as gene expression analysis, genotyping by hybridization (competitive hybridization and heteroduplex analysis), sequencing by hybridization, probes for Southern blot analysis (labeled primers), probes for array (either microarray or filter array) hybridization, “padlock” probes usable with energy transfer dyes to detect hybridization in genotyping or expression assays, and other types of probes. The DNA and RNA prepared in accordance with the invention may also be used in enzyme-based reactions such as polymerase chain reaction (PCR), as primers for PCR, templates for PCR, allele-specific PCR (genotyping/haplotyping) techniques, real-time PCR, quantitative PCR, reverse transcriptase PCR, and other PCR techniques. The DNA and RNA may be used for various ligation techniques, including ligation-based genotyping, oligo ligation assays (OLA), ligation-based amplification, ligation of adapter sequences for cloning experiments, Sanger dideoxy sequencing (primers, labeled primers), high throughput sequencing (using electrophoretic separation or other separation method), primer extensions, mini-sequencings, and single base extensions (SBE). The DNA and RNA produced in accordance with the invention may be used in mutagenesis studies, (introducing a mutation into a known sequence with an oligo), reverse transcription (making a cDNA copy of an RNA transcript), gene synthesis, introduction of restriction sites (a form of mutagenesis), protein-DNA binding studies, and like experiments. Various other uses of DNA and RNA produced by the subject methods will suggest themselves to those skilled in the art, and such uses are also considered to be within the scope of this disclosure.

Flow-Through DNA Synthesis Chip

The microarray apparatus of the invention allows for nucleotide oligomers to be synthesized on or proximate to porous regions of a substrate element of the apparatus. The apparatus comprises, in many embodiments, a substrate with a plurality of isolated porous regions extending through the substrate, which may be made by techniques such as photolithography, dry/wet etching process, ion or electron beam lithography, or other microfabrication process.

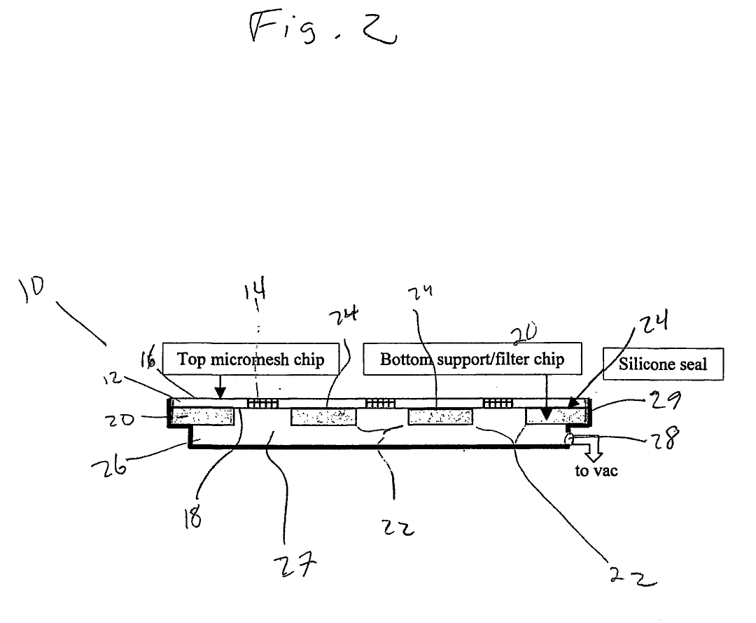

Referring to FIG. 1 and FIG. 2, there is shown a chemical synthesis apparatus 10 in accordance with the invention. The apparatus 10 in FIG. 1 comprises a substrate element 12 with isolated porous or mesh regions 14 which extend through the substrate 12, allowing reagents, fluids and the like to flow from the upper or first surface 16 of the substrate element 12 to the bottom or second surface 18 of the substrate element 12. Porous regions 14 thus extend through substrate 12 and communicate with the upper and lower surfaces 16, 18 thereof to allow passage of reagents therethrough as described further below. Each porous region 14 may comprise any porous material, and includes a plurality of pores, channels or openings (not shown) that allow fluid flow through the porous region.

The apparatus 10 further comprises a second substrate or back element 20 that includes an array of holes 22. The back element 20 is coupled to the bottom or second surface 18 of the substrate 12 such that each of the holes 22 of the back element 20 align with corresponding ones of the porous regions 14 of the substrate 12, to allow reagents to flow freely to and from the porous regions 14 of the substrate 12 through the holes 22 in the back supporting element 20. Juxtaposed between the substrate 12 and back element 20 is a seal 24, which also includes a corresponding plurality of holes or openings 25 to match the porous regions 14 of substrate 12 and holes 22 of back element.

A holder or enclosure 26 is provided that is configured to support back element 20 and substrate 12 to provide a sealed enclosure 27 that is in flow communication with porous regions 14 of substrate 12. A pressure differential across substrate 12 may be introduced by application of vacuum to enclosure 27, or by introducing pressure outside enclosure 27, by a vacuum source (not shown) or other means for generating a pressure differential across substrate 12. A vacuum channel 28 allows attachment of a vacuum source to provide such a pressure differential. The change in pressure allows fluid flow through porous regions 14, and allows fluids to be directed into and out of the porous regions 14 of the substrate 12 as well as the array of holes 22 of the back supporting element 20. The support element 20 provides mechanical support for the substrate 12, and the holes 22 in substrate 20 direct filtrate from the porous regions 14 of the substrate 12 to enclosure 27. The apparatus 10 may further comprise an absorbent element 30 positioned in holder 26 to absorb and trap used reagents during the various steps in oligomer synthesis as described further below.

The holder 26 maybe manufactured from materials and compositions which are inert to solvents and reagents utilized in oligomeric synthesis, such as anodized aluminum or other metal or metal alloy and/or polymeric materials. Various polymeric material, such as PEEK (polyether-ether-ketone), polypropylene, polyether sulfone and like materials may be used. Holder 26 may comprise a single integral component that is molded from the same material.

The substrate 12 and support 20 of the microarray apparatus of the invention may be fabricated from a variety of compounds such as silicon, glass, ceramics, ferrous or non-ferrous alloys or organic or inorganic polymers. Examples of organic or inorganic substrate compositions include, but are not limited to, polyolefins, polyimides, fluorocarbon polymers, polyetheretherketones, polyamides and polysiloxanes. The substrate 12 may also be fabricated from composite material such as those obtained by combinations of the materials mentioned above. The isolated porous regions 14 and holes 22 are formed by etching or microfabrication techniques as described below.

In many embodiments, the substrate 14 and the support or back element 20 may be fabricated from silicon wafers, such that porous regions 14 and holes 22 can be formed by conventional silicon microfabrication techniques. In FIG. 2, porous regions 14 are shown as silicon micromesh grids, with the holes 22 of the support element 20 positioned align with the grid regions 14 such that the approximate center point of each hole 22 is in registry with the grid regions 14 of the substrate 12. The substrate 12 and support element 20 are coupled together by silicone seal 24, allowing them to function as a single substrate/support component. In other embodiments, porous regions 14 may comprise controlled pore glass regions within a glass, ceramic, plastic or metal plate as a substrate. Numerous other porous materials and substrates for supporting porous materials may be used with the invention as well.

In certain embodiments, silicone seal 24 comprises a coating of material such as ZIPCONE UA™ which is an acrylic-modified silicone that is photodefinable to allow formation of holes 25 through seal 24. The silicone seal 24 is fabricated by spin-coating a layer of ZIPCONE UA™ onto the support element 20, which is then irradiated with UV (˜250 nm) through a negative of the physical mask used to form holes 25 in seal 24, and allow subsequent etching of the holes 22 in the support element 20. The relatively smooth silicone layer 24 is thus formed on the support element 20 except on the through-holes 22 which remain open. This silicone layer 24 on the support wafer 20 provides a good seal between support 20 and the substrate 12 and provides for abrogating any lateral spreading of reagent solutions between the substrate 12 and support element 20.

The grid or porous regions 14 are the regions of the substrate 12 where molecules of interest, such as oligonucleotide chains, are anchored and synthesized. The pores of grid regions 14 of the substrate 12 may have a diameter ranging from between about 0.1 millimeter in diameter and about 1 millimeter in diameter, between about 0.1 millimeter in diameter and about 10 micron in diameter, between about 1 micron in diameter and about 10 micron in diameter, between about 1 micron in diameter and about 100 nanometers in diameter, and between about 100 nanometer in diameter and about 1 nanometer in diameter. The size of the individual pores, perfurcations or wells (not shown), as well as the overall size of each porous region and the density of porous regions on substrate 12, will vary depending on the intended use of the invention.

Using photolithographic techniques to fabricate the apparatus 10 allows for a high density array of grid or porous regions 14 per surface area of substrate 20. The number of porous or grid regions 14 on the substrate 12 may range from about 1 to about 10 regions per cm², about 10 to about 100 regions per cm², about 100 to about 1000 regions per cm², about 1000 to about 10000 regions per cm², about 10000 to about 100000 regions per cm², and in certain embodiments the porous regions 14 may be present on substrate in a surface density of about 100000 to about 10000000 grid or porous regions per cm². Silicon microfabrication techniques offer increasingly fine resolution and may be able to provide even smaller pore sizes and higher densities of porous regions in the future.

In certain embodiments of the invention, substrate 10 and porous regions 14 may be configured to conform to the microplate formats described by the Society for Biomolecular Screening (SBS), http:/www.sbsonliine.org/disgrps/platestd/details.html. The SBS microplate formats include a 96 well plate, a 384 well plate and a 1536 well plate each have a dimension of approximately 5.03 inches by 3.365 inches, or approximately 16.926 square inches. This corresponds to a density of approximately six porous regions per square inch for a 96 well plate, approximately 23 porous regions per square inch for a 384 well plate, and approximately 93 regions per square inch for a 1536 well plate. More preferably, the apparatus of the invention provide a porous region density of greater than approximately six porous regions per square inch in a 96 well plate format. In a 384 well format, the apparatus of the invention may have a porous region density of greater than approximately 23 porous regions per square inch. In a 1536 well plate format, the apparatus of the invention preferably have a porous region density of greater than approximately 93 porous regions per square inch.

A silicone seal or gasket 29 may be utilized to insure a tight seal between the holder 26, substrate 12 and support 20 element as shown in FIG. 2. In some embodiments, the substrate 12 and support 20 may be fused to the holder 26 to provide a seal, allowing introduction of vacuum to enclosure 27 such that liquid and used reagents can flow through the porous regions 14 and the holes 22 of the substrate 12 and support 22 respectively.

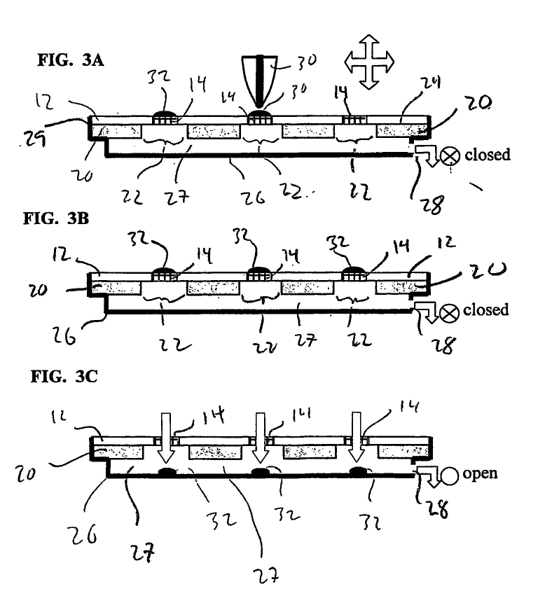

Referring to FIG. 3A through FIG. 3C, the use of the apparatus 10 for synthesis or coupling of molecules of interest onto grid elements 14 of substrate 12 is illustrated. In FIG. 3A, a dispenser head 30 is used to apply droplets 32 of reagent selectively onto porous regions 14 of substrate 12. Different reagents may be present in different droplets so that different reagents are presented to selected ones of the porous regions 14. Dispenser head 30 may be part of a multiple head dispenser system (not shown) as described further below. Movement of dispenser head 30 with respect to the apparatus 10 may be carried out by translation of dispenser head 30 with respect to the apparatus 10, translation of the apparatus 10 with respect to a stationary dispenser head, or both.

In FIG. 3B a reagent droplet 32 is shown on each of the porous regions 14. The droplets 32 are allowed to remain in contact with porous regions 14 as long as required in order to allow bonding or coupling of reagent within droplets 32 to the surface of porous regions 14 (and/or to the surface of substrate 12 adjacent to porous regions 14). The surfaces of porous regions 14 may be chemically modified prior to application of droplets 32 to facilitate bonding of reagent thereto, as described further below.

In FIG. 3C, vacuum is applied to the interior enclosure 27 of holder 26 via vacuum port 28, to draw reagent droplets 32 through porous regions 14 and holes 22 in support 20 into enclosure 27. An absorbent layer (not shown) may be included therein to absorb unreacted reagent in the droplets 32. The events illustrated in FIG. 3A-3C may be repeated as required, using droplets 32 containing various reagents, to selectively form oligomeric molecules of desired compositions at each of the porous regions 14.

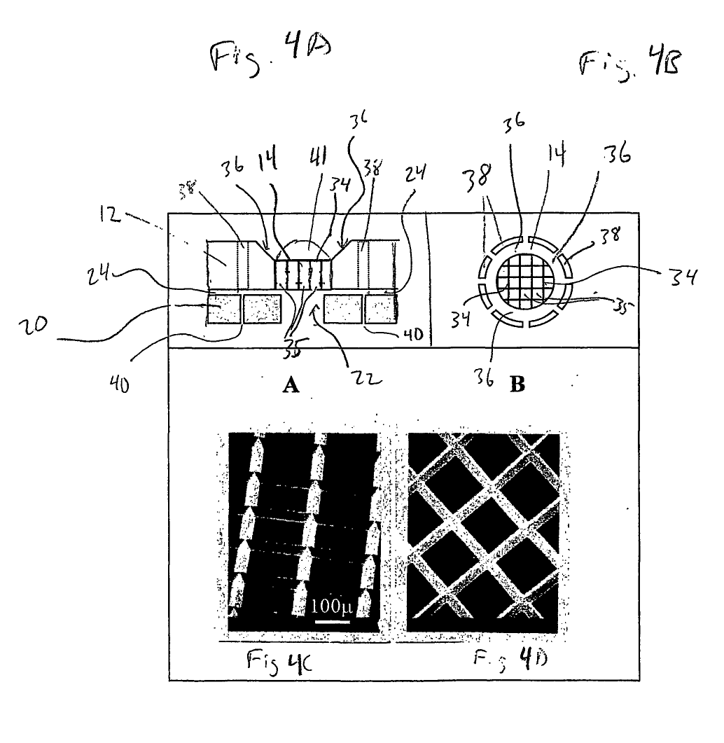

Referring now to FIG. 4A and FIG. 4B, there is shown a detail of an individual porous region 14 in one embodiment of the invention, together with the adjacent portions of substrate 12 and support 20. The porous region 14 comprises a mesh 34 with a plurality of pores 35 extending therethrough. The porous region 14 is recessed with respect to the top surface 16 of substrate 20 such that a wall or barrier 36 surrounds each porous region 14. A plurality of channels or holes 38 through substrate 20 are arranged around porous region 14 outside of wall 36 to act as a “moat”. Channels 38 and/or barrier 36 may be omitted in some embodiments of the invention. Matching holes or channels 40 may be included in support 20 which are positioned to communicate with channels 38 in substrate 20. Porous region 14 is shown in FIG. 4A with a droplet or microdroplet 41 of reagent thereon. Wall 36 and channels 38 serve as barriers that prevent cross-contamination of reagent 41 between adjacent porous regions 14 in the apparatus 10 during synthesis of oligomers or other molecules at porous regions 14.

The depth or amount of recess of the porous grid region 14 with respect to the top surface 16 of substrate 12 may vary in different embodiments of the invention. Mesh 34 may be recessed from surface 16 by about 0 to about 10 mm in certain embodiments, from 0 to 1 mm in some embodiments, from about 10 to about 500 microns in other embodiments, and from about 40 to about 60 microns in still other embodiments.

FIG. 4C and FIG. 4D are electron microscopy photographs of a portion of a porous region 14 of substrate 20 that clearly shows the mesh 34 and interstitial pores 35 within the mesh 34. The mesh pattern of porous regions 14 may be formed by coating photoresist on each surface 16, 18 of substrate, suitably patterning the photoresist to define the mesh 34 and pores 35, and using wet chemical etching techniques to form pores 35. After wet chemical etching to form the porous regions 14 in substrate 12, the surface 16 of substrate 12 is oxidized to provide a uniform coating of SiO₂ thereon, including the mesh 34 and the adjacent portions of wall 36 for each porous region 14. Wet chemical etching techniques for formation of structures in silicon, as well as surface oxidation of silicon substrates, are well known in the art. The grid element 14 shown in FIGS. 4C and 4D has square pores 35 with a pore size of approximately 100 micron per side.

The entire surface 16 of the substrate 20 may then treated with a perflourinated silyl chloride compound to provide a covalently attached fluorocarbon surface which is both hydrophobic and oleophopbic and is poorly wetted by aqueous solutions and most common solvents. An exemplary surface treatment compound is a chlorinated fluoroalkylmethylsiloxane such as AQUAPHOBE CF™. Although the contact angle for solvents like acetonitrile and methylene chloride (common solvents used in the DNA synthesis) are not as great as that for water on AQUAPHOBE CF™, the solvents tend to bead up and do not wet the surface appreciably. The porous grid elements or regions 14 however, are cleaned of this surface polymer by a brief treatment in an oxygen plasma to provide the SiO₂ surface necessary for the covalent attachment of the growing oligonucleotide. The application of fluoroalkylmethylsiloxane coating onto silica surfaces, and use of oxygen plasma to selectively remove portions thereof, are described in U.S. Pat. No. 5,474,796, the disclosure of which is incorporated herein by reference.

Each grid element or porous region 14 is thus configured to localize a liquid reagent drop 42. The surface treatment with fluoroalkylmethylsiloxane coating, as well as the fact that the grid region 14 is recessed from the top surface 16 of the substrate 12, both prevent the lateral spread of the reagent drop 42 into adjacent grid wells (not shown). The recessed grid 14 shown in FIG. 4A is possible because of the fact that the substrate 12 is patterned with photoresist on both sides during formation of mesh 34 and pores 35, with the two patterns aligned with the aid of an infrared mask aligner.

In the formation of molecules of interest on the porous regions 14 of substrate 12 of the apparatus 10, drops of reagent are applied to selected ones of the porous regions and allowed to incubate or otherwise remain in contact with the porous regions 14 so that a reaction can occur to introduce a molecule of interest, or a precursor or linker therefore, to the porous regions 14. Once the reagent drop 42 has incubated for a sufficient amount of time to allow a component in the liquid drop to interact with the grid element 14, or materials bound to the grid element, the liquid is drawn through the grid element 14 by initiating a pressure difference across the substrate 12 and support 20. The support element 20 provides mechanical support and appropriately directs the flow of the used reagent fluid away from the porous grid elements 14, with the array of holes 22 in registry underneath each flow-through grid elements 14 in the substrate 12. In certain instances, the used reagents may be immobilized in an absorbent medium 30 in holder 26 (as shown in FIG. 1).

Fabrication of DNA Synthesis Apparatus

The manufacturing techniques used to prepare the apparatus 10 are well established and known to those skilled in the art. The fabrication of the pores or perforations 35 into the substrate 12 may be completed by methods such as microfabrication, micromachining or MEMS (micro electromechanical system), wet chemical etching, ion bombardment, reactive ion etching, water jet, mechanical cutting, abrasion or drilling and the like. In certain embodiments, the grid patterns of regions 14 maybe etched into the wafer by first growing or depositing a layer of silicon dioxide, or other suitable material on the surface of the wafer 12, followed by coating the wafer with photoresist and defining the desired grid pattern onto the photo resist coating on both faces of the wafer 12, transferring the resist defined pattern in to the oxide with a suitable oxide, such as fluorine containing plasma or hydrofluoric acid containing mixtures, then etching the expose silicon with a basic solution.

In one embodiment, the intricate pattern of the grid regions 14 was achieved by having a photoresist pattern marked on both the oxidized upper and lower surfaces 16 and 18 of the substrate 12. A commercially available double side exposure system was employed which allowed the alignment of the top and bottom photoresist patterns relative to each another. The developed resist patterns were used to mask the oxide from a commercially available premixed ammonium fluoride—hydrofluoric acid based buffered oxide etch (BOE). The resist layer, was then removed with suitable solvents, followed by a hot sulfuric acid-oxidizer clean. The silicon substrate 12 was then etched along the <100> surfaces with a strong base treatment that does not rapidly attack the photoresist, the oxide passivation layer, or the <111> crystal planes on the surfaces 16 and 18 of the substrate 12. Since a solution based etch was used, the etching occurred from both surfaces 16, 18 of the substrate 12 simultaneously. Many base solutions are known for based etching, and an exemplary solution useful in fabricating the grid meshes 14 is a solution of KOH (5-10 Molar) in aqueous isopropyl alcohol at 40-80° C., preferably 70° C. to 80° C. Grid regions 14 range in thickness depending on the thickness of the substrate. Grid thicknesses of 1-1000 microns are feasible with the above techniques. It is an extremely cost effective process as the reagents are very inexpensive and there is no practical limit to parallel processing many wafers simultaneously.

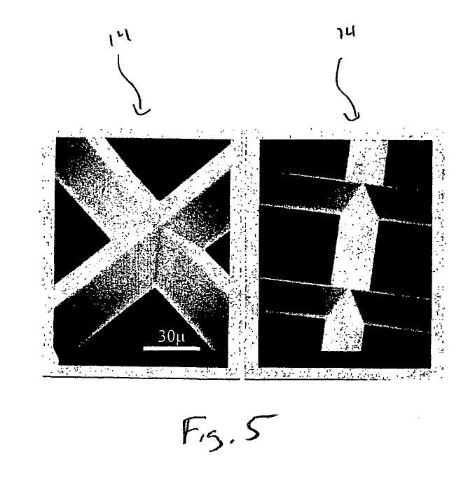

FIG. 5 is a pair of scanning electron microscopy photographs of the etched micromesh grids 14 on a silicon substrate prepared by a wet etch method as described above. The four-fold symmetry of the grid 14 reflects the fact that the Si [100] direction is oriented perpendicular to the surface of the substrate. The angle between the etched edges of each mesh element and the wafer surface is approximately about 54.7°, which corresponds to the angle between the [100] and [111] planes in the silicon crystal lattice. The size of the grid elements 14 shown in FIG. 4 are only exemplary and grid elements of various sizes can be achieved by the described fabrication methods, as noted above. The features on the grid 14 do not require that they be micromachined perpendicular to the surface, thus the micromachining operation can be performed by the wet chemical etching technique. The surface of these grid elements 14 can be textured on a very fine length scale to increase the surface area if desired. Surfaces areas of three to five times that of a flat surface can be achieved with judicious texturing. The size or surface area of the individual grid elements 14 may range from about 0.1 micron to about 1,000,000 microns in diameter, more preferably from about to about 5 microns to about 100,000 microns, and even more likely to range from about 10 to about 1000 microns.

The method of fabrication of the apparatus of the invention varies from conventional DNA chip making methods by using photolithography techniques only to prepare the chip, and not during oligonucleotide growth/synthesis. The large numbers of complex photolithographic steps normally necessary for DNA growth on a chip are not required by the present invention, since no photolabile protecting groups are utilized in the DNA synthesis.

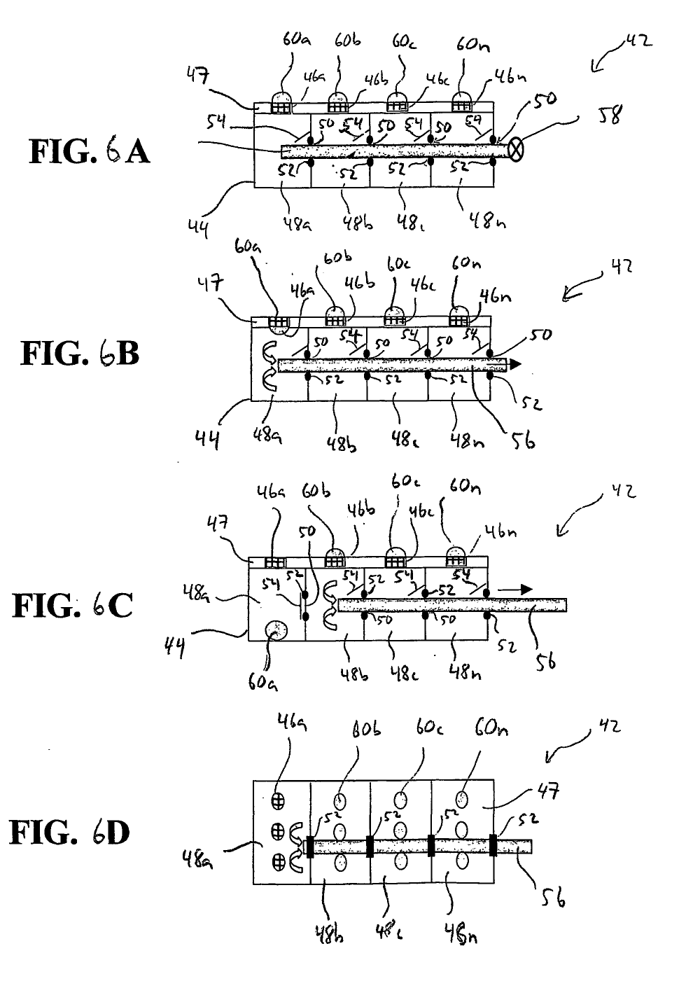

Referring now to FIG. 6A through FIG. 6D, another embodiment of a microarray apparatus 42 is shown. This embodiment allows for the staging of reagent removal with respect to time. The apparatus 42 as shown in FIG. 6A comprises a holder 44 configured to support a substrate 47 comprising grid regions 46a, 46b, 46n that extend through substrate 47 and communicate with the interior of holder 44. A support element (not shown) may be included under substrate 47 for additional support thereof. The interior of holder 44 comprises a plurality of compartmentalized regions 48a, 48b, 48c and 48n that are respectively adjacent grid elements 46a, 4b, 46c, 46n. Each compartment 48a, 48b, 48c may be associated with a single grid element or multiple grid elements. As shown in FIG. 6D, a row of grid elements 46a are associated with compartment 48a, with each compartment 48b, 48c, 48n being associated with a corresponding row of grid elements. Alternatively, each row of grid elements 46a-46n could be serviced by a solenoid which could evacuate a given row of the device at the prerequisite time.

Each compartment 48a, 48b, 48c, 48n includes an opening 50 surrounded by a seal 52. Each opening 48a, 48b, 48c, 48n also includes a hinged gate 54, which may be biased towards a closed position wherein gate 54 engages seal 52. Openings 50 are configured to accommodate a hollow tube element 56 that is connected to a vacuum source 58. Tube 56 slidably engages each opening 50 and contacts seals 52 in openings. When tube 56 fits through an opening 50, the hinged gate 54 associated with that opening is pushed into an open position, as can be seen most clearly in FIG. 6A and FIG. 6B. When tube 56 is withdrawn from an opening 50, the hinged gate 54 associated with that opening returns to a closed position and engages the seal 52 surrounding the opening 50, as shown for the gate 54 of compartment 48a in FIG. 6C.

The compartments 48a-48n of holder 44 are constructed so that the evacuation tube 56 is limited to communicate with only the grid elements that are in contact with one given compartment at any time. Thus, a vacuum can be selectively applied to one of compartments 48a-48n, while the other compartments remain at ambient or atmospheric pressure. The compartments 48a-48n can sequentially evacuated as the tube 56 is withdrawn from openings 50 such that the open end of tube 56 is positioned within a selected compartment. In this manner, only the grid elements 46a-46n situated above a particular row address on the array communicate with a pressure change via vacuum source 58. The apparatus 42 allows the user to precisely and automatically control the amount of time that reagent contacts grid elements grids 46a-46n.

In the use of apparatus 42, synthesis of DNA oligomers or other molecules of interest is initiated by dispensing liquid droplets 60a, 60b, 60c, 60n onto the isolated micromesh grid regions 46a, 46b, 46c, 46n respectively by a dispenser head (not shown). Each droplet 60a-60n may comprise a different reagent to provide for synthesis of a different oligomer at each grid site 46a-46n. Selective application of droplets 60a-60 may be carried out by moving the apparatus 42 (which may be fixed to a translation stage, not shown) with respect to the dispensing head, moving the dispensing head with respect to the apparatus 42, or both. In certain embodiments, a translation stage that moves in the x-direction, and a dispensing head which moves in the y direction, may be used for selective application of reagent droplets 60a-60n onto grid regions 46a-46n.

The evacuation tube 56 is inserted into the compartment 48a-48n above which the reagents 60a-60n have been applied. As shown in FIG. 6A and FIG. 6B, the tube 56 has passed through all of the “trap-doors” 54 and the end of tube is pressure isolated within compartment 48a from the adjacent compartments via o-ring seals 52. As vacuum source 58 is opened or applied, the selected compartment 48a evacuates, and the reagent droplets 60a on the grid elements 46a are drawn through the grid elements 46a into compartment 48a as shown in FIG. 6B and FIG. 6C. Compartments 48a-48n each may contain an absorbent charcoal reagent waste receptacle (not shown).

In order to access the next compartment 48b, vacuum source 58 is closed and the tube 56 is withdrawn from the opening 50 between compartments 48a, 48b, as shown in FIG. 6B and FIG. 6C. Tube 56 may be moved by withdrawing the tube itself, or by translating the apparatus 42 with respect to a stationary tube. As the evacuation tube 56 passes through the opening 50, the gate or door 54 closes behind the exiting tube 56 and engages seal 52. When vacuum source 58 is again opened or applied, the gate 54 seats firmly on the o-ring seal 52 due to the pressure differential (the originally accessed compartment 48a rapidly vents to atmospheric pressure after the door 54 is closed). The process is repeated in compartment 48b, 48c and on to compartment 48n. The timing of removal of reagent droplet 60a-60 from each grid element 46a-46n may be carefully controlled by use of apparatus 42 in the above-described manner.

The use of apparatus 42 requires only one type of motion control. The apparatus 42 may be attached to a stage that moves in the ±x direction, while a dispense head moves in ±y direction. The tube placement into the holder 44 of apparatus 42 thus may be controlled solely by the x motion of the stage. The time that the reagent drop 60a-60n contacts the grid element 46a-46n can be controlled by the rate at which the evacuation tube 56 is moved relatively to the holder 44. Motion control may be provided by a simple x-y translation stage to which the apparatus 42 is affixed and/or a similarly translatable dispenser head. Additional motion control beyond that of the liquid dispenser's stage and the stage motion can be easily programmed into commercially available liquid dispenser systems, and no additional software or hardware is necessary.

Oligomer Synthesis Methods

The apparatus of the invention is configured to provide a liquid handling system and a system for generating a pressure difference across substrate containing porous regions. Peripheral components utilized in the methods of synthesizing oligomers described below include a liquid dispensing system comprising a printhead and its corresponding motion control, a reagent pack and its associated fluid handling, a translation stage and holder for the apparatus, and a vacuum/inert gas pressure control system.

Liquid Handling System—The apparatus may further comprise various types of liquid dispensing systems for applying reagents to grid or porous regions on a substrate. A four head liquid dispenser such as a synQuad™ from Cartesian Technologies is an exemplary liquid handling system, and was used in the examples below. This four head system is capable of filling −25 wells/sec by dosing “on-the-fly”, which translates into the filling of a 384 plate in −15 seconds and a 1536 plate in under 60 seconds.

In some embodiments, the delivery of synthesis reagents and wash solutions to the substrate may be carried out by a 10-port piezoelectric printhead which is commercially available from such sources as Microfab, Inc. The action of the printhead is controlled by printhead controlling software that allows the modification of the action of the printhead and the dispensing of specific reagents depending on the type of oligomeric synthesis to be completed. The printhead is attached to an x-y motion control assembly, and the precision x-y motion control hardware is used to program the printhead movement in order to synchronize the dispensing of the correct chemical with the location of the printhead on the substrate as well as triggering the pressure change (vacuum) needed for reagent removal.

In other embodiments, the liquid handling system may be configured to dispense solutions using a disposable reagent pack, including for example in the case of DNA synthesis, a reagent pack comprising the phosphoramidite reagents for the nucleic acids G, C, T, A, and any necessary wash solvents. With the entire reagent volume employed to the array of grids or porous regions is approximately 500 μL divided among 10 reagents, each vessel in the reagent pack will only need to hold less than 200 μL in volume. Reagent pack containers for phosphoramidite reagents for the nucleic acids G, C, T, A, possessing small compression type fittings to a tubing which easily fits into a small region of the device enclosure, are commercially available from Upchurch Scientific, Inc. The reagents packs will be in pressure equilibrium with the inert gas atmosphere on the inside of the enclosure as the reagents are dispensed so as not to create a vacuum inside the reagent dispensing vessels.

The volume of liquid reagent applied to or contacted with each grid or porous region may vary in different embodiments of the invention. Application of liquid reagent to each porous region may comprise, for example, applying a liquid reagent having a volume of between about 1 milliliter and about 100 microliters in volume, a volume of between about 100 microliters and about 1 microliter, a volume of between about 1 microliter and 100 nanoliters, a volume of between about 100 nanoliters and 1 nanoliter, a volume of between about 1 nanoliter and about 1 picoliter, or a volume of between about 1 picoliter and about 1 femtoliter.

Many systems for selective application of liquid reagent to array substrate surfaces are known and may be used with the invention. Additional liquid reagent dispenser systems usable with phosphoramidite-based reagents and other biomonomer reagents are disclosed in U.S. Pat. Nos. 5,474,796 and 5,449,754, the disclosures of which are incorporated herein by reference.

Pressure Control Systems. Pressure differential across the apparatus substrate may be provided by a conventional vacuum pump and connection hose. Movement of reagent drops 60a-60n through grid regions 46a-46n may alternatively be carried out via other mechanisms, including mechanical, shape memory alloy, electrostrictive material or piezoelectric actuated pumps and/or solenoids. The fluid reagent droplets 60a-60n may also be moved by osmotic pressure, electroosmotic pressure, electric fields, capillary action, gravity, or other mechanism or effect.

The water and air sensitivity of some of the DNA synthesis reagents, in particular the phosphoramidites, require that the synthesis reactions be run under an inert atmosphere. Also, once the pressure is reduced within the enclosed region underneath the substrate containing grid regions 46a-46n, an injection of “make up” gas may be required to main the pressure above the grid regions 46a-46n at ambient pressure. This may be accomplished by connection of a small, pressurized container of inert gas. The system pressure may be monitored, and the gas added as required, using the pressure sensor/regulator of TiNi Alloy Company of San Leandro Calif. These pressure sensor/regulators are based on the use of thin titanium-nickel alloy films that are shape memory materials used for the mechanical actuation. The Ti:Ni 1:1 films can be cast into a given shape, after which they are deformed or stressed into a desired shape. When the cast films are heated above their martensitic transition temperature, they revert to their original shape, exerting pressure of up to 50,000 psi. Some of these films have been stressed and recovered nearly 109 times.

In some embodiments, a second valve or solenoid may be used for the actuation of the vacuum system. Furthermore, it is necessary for the triggering of the vacuum valve to be synchronized with the dispensing timing. The grid elements 46a-46n on each chip are filled by the dispenser within about 10 seconds, then after a suitable reaction time, the vacuum valve is actuated and the used reagent drawn through the grid and support/filter wafer into an absorbent medium for immobilization. Software is implemented to synchronize the above mentioned acts.

In still other embodiments, a combination of gas and pressure may be used to move the liquid reagent 60a-60n through grid regions 46a-46n. There are a large variety of small vacuum pumps that are commercially available which are suitable for the oligomeric synthesis methods of the present invention. An exemplary pump is one from PAR Technologies that is approximately 1″×1″×0.25″ in size (0.25 in² volume), is chemically inert and can run for a week on a 9V battery. This pump is actuated with a piezoelectric element noise and vibration are nearly non-existent.

The reagent vessels used in DNA synthesis have to obtain a pressure equilibration to function after dispensing. The pressure may be controlled by employing miniature valves and regulators (footprint of 25-100 mm²) made with TiNi shape memory alloy actuators that are commercially available.

Software and Programming—The apparatus of the invention may further comprise software programs which provide control for the synchronization of the dispense/vacuum/refill functions such as synchronizing the printhead movement, dispense action and vacuum actuation. The software is programmed to interface between the user specified base sequence and the position of the dispense head to deliver the correct reagents to specified grid regions. For DNA synthesis by phosphoramidite chemistry, the program comprises a four color printing algorithm since the capping, oxidation, deprotection and washing steps are the same for each grid element and only the step which involves dispensing of one of the four nucleotides is different for individual grid regions.

DNA Synthesis Chemistry

For the synthesis of DNA oligomers by the methods of the invention, traditional phosphoramidite chemistry may be carried out for the DNA chain growth portion of the synthesis. Phosphoramidite based chemical synthesis of nucleic acids is well known to those of skill in the art, being reviewed in Streyer, Biochemistry (1988) pp 123-124 and U.S. Pat. No. 4,415,732, herein incorporated by reference. Phosporamidite reagents, including B-cyanoethyl (CE) phosphoramidite monomers and CPG (controlled porous glass) reagents usable with the invention may be purchased from numerous commercial sources, including American International Chemical (Natick Mass.), BD Biosciences (Palo Alto Calif.), and others. The chemicals used for the oligonucleotide synthesis are known for their rapid and nearly quantitative reactivity. Even in a DNA synthesizer, where the growing nucleotide chain is attached to the internal pores of the CPG glass, which are less accessible that the totally exposed surface of the grid, the reaction goes to >99% completion in 5-30 seconds.

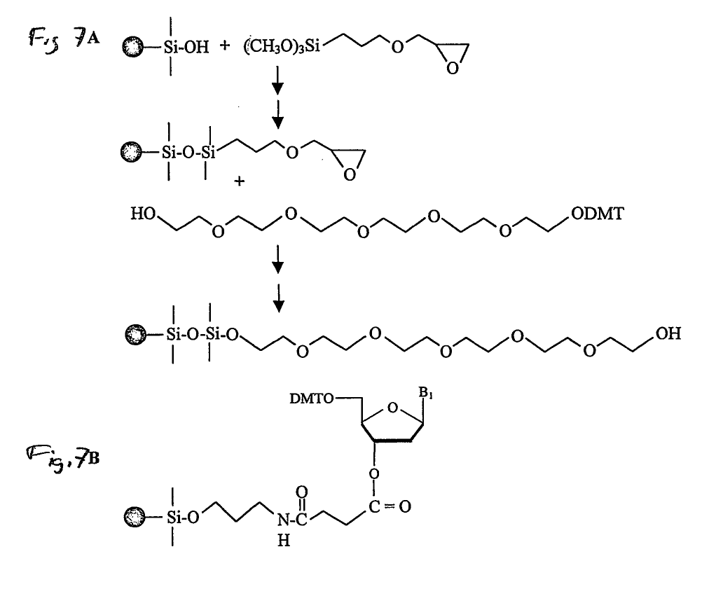

In order to use phosphoramidite chemistry, the surface of the grid regions 46a-46n of the substrate 47 should be chemically modified to provide a proper site for the linkage of the growing nucleotide chain to the surface. Various types of surface modification chemistry exist which allow a nucleotide to attached to the grid surface. The type of grid surface modification implemented depends on whether one wants to cleave the oligonucleotide chain from the surface concomitant with deprotection of the nucleic acid bases, or leave the nucleic acid chain attached to the grid element after deprotection. One surface modification technique that allows for the exocyclic N atoms of the A, G and C bases to be deprotected while having the oligonucleotide chain remain attached to the substrate as shown in FIG. 7A. This chemistry is well known and is described at http://www.glenresearch.com/and incorporated herein by reference.

Another scheme of reacting a trialkoxysilyl amine (e.g. (CH₃CH₂O)₃Si-(CH₂)₂-NH₂) with the glass or silica surface SiOH groups, followed by reaction with succinic anhydride with the amine to create and amide linkage and a free OH on which the nucleotide chain growth could commence, is shown in FIG. 7B

A third type of linker group may be based on photocleavable primers. The advantages of this type of linker is that the oligonucleotide can be removed from the substrate (by irradiation with ˜350 nm light) without cleaving the protecting groups on the nitrogenous functionalities on each base. The typical ammonia or NH₃ treatment deprotects everything when used as the reagent to cleave the oligomers from the substrate. The use of photocleavable linkers of this sort is described at http://www.glenresearch.com/, noted above. Various other cleavable linker groups may alternatively be used. Cleaving of the linker group provides usable DNA that may be used in the manner described above.

Method of Synthesizing DNA

Referring again to FIG. 3A-3C, one embodiment of the method of synthesizing oligomers onto the apparatus of the invention can be seen. A multiport piezoelectric ink-jet, or any other type of liquid dispenser 30 is positioned to place a drop 32 of a selected DNA synthesis reagent onto an individual grid element 14. The reagent drop 32 is allowed to incubate on the grid element 14 for an appropriate length of time. After a suitable reaction time, the drop 32 is removed from the grid reaction zone 14 by reducing the pressure on the underside of the substrate 12, allowing the reagent liquid 32 to flow-through the grid region 14 of the apparatus 10 into enclosure 27. The used reagent flows through the holes 22 in support element 20 and is immobilized in an absorbent medium 30 (shown in FIG. 1). The removal of the used reagent is followed by the addition of a wash reagent to the upper surface of the grid region 14, provided from another channel of the liquid dispenser, after which yet another channel of the multiport dispenser device delivers the reagent for the next step of the oligomer synthesis.

The method of synthesizing DNA oligomers with the apparatus of the invention may involves the priming of the grid elements 14 for attachment of the oligonucleotides. The surface of the grid elements 14 may be chemically modified to provide a proper site for the linkage of the growing nucleotide chain to the surface. The surface treatment starts with an oxidation step of treating the as-fabricated silicon mesh surface with moist air at 1000° C. overnight to grow a dense a strongly adherent SiO₂ surface of about 1,000-10,000 Å thick on the grid element surfaces. This treatment results in surfaces that are terminated in Si—OH groups. Trialkoxysilyl amine reacts with the silica surface SiOH groups, followed by reaction with succinic anhydride, forming an amide linkage and a free OH on which the nucleotide chain growth can occur.

After priming the grid elements 14 in the above manner, a first phosphoramidite nucleotide reagent is applied to selected grid regions 14. After approximately 5-20 seconds, the pressure is reduced slightly on the under side of the substrate 12 and the spent reagent passes through the grid 14, through the support element 20 and is immobilized on an adsorbent layer 30. Appropriate reagents for washing, capping, oxidation, and detritylation are dispensed and removed in a similar manner as the first phosphoramidite nucleotide reagent. The cycle time for the addition of one base (and appropriate subsequent washes and modifications) is about 2-3 minutes giving an overall time for oligomer synthesis as approximately 34 hours for the production of at least 100 distinct and arbitrary 70-base oligonucleotides on a substrate with an approximate size of 30 mm×30 mm×1 mm.

The length of nucleotides prepared is directly dependent on the yield of each step in the synthesis. For example, when synthesizing a 70-mer, one would get −50% yield of the final oligomers if each step was 99% efficient, but the total yield would rise to 70% if the step yield could be increased to 99.5%. Furthermore, unlike other reagent/dispensing removal systems used with conventional chip-based DNA synthesis, the present invention makes it possible to dose each grid array element more than once with the same reagent, increasing the yield of the reaction and the end product on the grid element.

The surface area of a textured grid is approximately that of a flat surface of an area of a conventional array region of similar diameter. The quantity of DNA synthesized in the apparatus of the invention is approximately 50-300 femtomoles/grid with a diameter of 500μ. A commonly accepted figure for the amount of DNA on the surface of a typical support is approximately 40 femtomoles/mm², indicating that the amount of DNA synthesized on the grid chips of the invention are quite comparable to that in other planar microarrays.

The length of the DNA or other nucleic acid synthesized in accordance with the invention may be of between about 2 nucleic acid bases and about 100 nucleic acid bases in length, between about 100 nucleic acid bases and about 1000 nucleic acid bases, between about 1000 nucleic acid bases and about 10000 nucleic acid bases in length, or between about 10000 nucleic acid bases and about 100000 nucleic acid bases in length.

Once an array of nucleic acid oligomers are formed on substrate 12, the array can have a variety of uses. Many such uses are disclosed in “Microarray Biochip Technology”, Mark Schena Ed., Eaton Publishing, Natick Mass. (2000), the disclosure of which is incorporated herein by reference. For example, target DNA molecules may be hybridized to the DNA oligomers on the substrate. The products or targets adhered to the oligomers attached to the microarray may be observed by many detection methods known to those skilled in the art such radioactivity, fluorescence, or by magnetic interactions. For example, a microarray for analyzing DNA samples may have a plurality of regions of defined features on which different probes are immobilized. The microarray is placed into a reaction container together with a fluorescence-labeled DNA sample or the like to allow the labeled DNA sample to hybridize with the probes immobilized on the respective features of the microarray. Thereafter, the microarray is irradiated with excitation light to measure fluorescent intensity of each feature. Based on the measured fluorescent intensities, the binding levels between the respective probes and the sample DNA are obtained and converted into desired information. This is just one type of microarray detection scheme and is not meant to limit the scope of the invention. Other detection schemes will suggest themselves to those skilled in the art.

The subject apparatus and methods can be used to synthesize other types of molecules of interest. The synthesis of peptides at selected grid regions is one such case. Various chemistries used in stepwise growth of peptides on an array surface are known. The peptide synthesis techniques described in U.S. Pat. No. 5,449,754, incorporated herein by reference, may be used with the present invention. The apparatus also finds uses in chemical synthesis of drugs, protein inhibitors or any chemical synthesis in which the rapid synthesis of a plurality of compounds is desired.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for the synthesis of an array of molecules, comprising:
(a) providing a substrate having first and second surfaces, and a plurality of isolated porous regions extending through said substrate and communicating with said first and second surfaces;
(b) contacting selected ones of said porous regions with a first reagent;
(c) allowing said first reagent to bind to said selected porous regions; and
(d) withdrawing unreacted said first reagent from said substrate through said selected porous regions by introducing a pressure differential across said substrate.

2. The method of claim 1, further comprising
(a) contacting said selected ones of said porous regions with a wash solution; and
(b) withdrawing said wash solution through said selected porous regions by introducing a pressure differential across said substrate.

3. The method of claim 1, further comprising:
(a) contacting said selected ones of said porous regions with a second reagent;
(b) allowing said second reagent to bind to first reagent at said selected porous regions; and
(c) withdrawing unreacted said second reagent from said substrate through said selected porous regions by introducing a pressure differential across said substrate;

4. The method of claim 3, further comprising
(a) contacting said selected ones of said porous regions with an nth reagent;
(b) allowing said nth reagent to bind to a previously bound said reagent at said selected porous regions; and
(c) withdrawing unreacted said nth reagent from said substrate through said selected porous regions by introducing a pressure differential across said substrate.

5. The method of claim 1, wherein said substrate includes at least 96 of said isolated porous regions.

6. The method of claim 5, wherein said substrate includes at least 384 of said isolated porous regions.

7. The method of claim 1, wherein said isolated porous regions comprise mesh regions etched into said substrate.

8. The method of claim 1, wherein said isolated porous regions comprise controlled porous glass regions associated with said substrate.

9. A method for the synthesis of an array of oligomeric molecules, comprising:
(a) providing a substrate having first and second surfaces, and a plurality of isolated porous regions extending through said substrate and communicating with said first and second surfaces;
(b) contacting selected ones of said porous regions with a first reagent including a first monomer;
(c) allowing said first monomer to bind to said selected porous regions;
(d) withdrawing excess said first reagent from said substrate through said selected porous regions;
(e) contacting said selected ones of said porous regions with a second reagent including a second monomer;
(f) allowing said second monomer to couple to said first monomer;
(g) withdrawing excess said second reagent from said substrate through said selected porous regions;
(ii) repeating events (e), (f) and (g) n times using n reagents with n monomers respectively, wherein n equals zero or an integer number, to form said array of oligomeric molecules.

10. The method of claim 9, further comprising contacting said porous regions with a surface modifier capable of allowing said first monomer to bind to said porous regions.

11. The method of claim 9, further comprising
(a) contacting said selected ones of said porous regions with a wash solution; and
(b) withdrawing said wash solution through said selected porous regions by introducing a pressure differential across said substrate.

12. The method of claim 9, wherein said oligomeric molecules comprise nucleic acids.

13. The method of claim 12, wherein said nucleic acids are selected from the group consisting of DNA and RNA.

14. The method of claim 9, wherein said oligomeric molecules comprise peptides.

15. The method of claim 12, further comprising applying a target nucleic acid molecule to said array of nucleic acids and allowing said target nucleic acid molecule to hybridize with said nucleic acids.

16. A method for hybridizing nucleic acids using the array of claim 12, comprising:
(a) applying a target nucleic acid molecule to said array of nucleic acids;
(b) allowing said labeled target nucleic acid molecule to hybridize with said nucleic acids; and
(c) washing said array to remove unhybridized labeled target nucleic acid molecule therefrom.

17. The method of claim 15, wherein said target nucleic acid is labeled.

18. The method of claim 17, further comprising detecting said labeled target nucleic acid molecule on said array.

19. The method of claim 18, wherein said labeled target nucleic acid includes a fluorescent label, and said detecting comprises fluorescence detecting.

20. The method of claim 18, wherein said labeled target nucleic acid includes a magnetic label, and said detecting comprises magnetic detecting.

21. The method of claim 6, wherein said surface modifier comprises a cleavable linker group.

22. A method for producing oligomers from the array of claim 21, comprising cleaving said cleavable linker group to release said oligomers from said substrate and form a plurality of free oligomers.

23. The method of claim 9, wherein said isolated porous regions each comprise a plurality of holes extending through said substrate, said holes plurality of holes formed by a microfabrication technique.

24. The method of claim 23 wherein said microfabrication comprises a technique selected from the group consisting of wet chemical etching, ion bombardment, reactive ion etching, water jet, mechanical cutting, abrasion, ion beam lithography, electron beam lithography, and drilling.

25. The method of claim 9, wherein said substrate material is selected from the group consisting of silicon, glass, ceramic, ferrous metal alloy, and non-ferrous metal alloy.

26. The method of claim 9, wherein said substrate material is a polymeric material selected from the group consisting of polyolefins, polyimides, fluorocarbon polymers, polyetheretherketones, polyamides and polysiloxanes.

27. The method of claim 9, where said withdrawing said excess reagent from said substrate through said selected porous regions comprises introducing a pressure differential across said substrate.

28. The method of claim 9, where said withdrawing said excess reagent from said substrate through said selected porous regions comprises use of osmotic pressure.

29. The method of claim 28, where said withdrawing said excess reagent from said substrate through said selected porous regions comprises use of electro-osmotic pressure.

30. The method of claim 9, where said withdrawing said excess reagent from said substrate through said selected porous regions comprises use of electric fields.

31. The method of claim 9, where said withdrawing said excess reagent from said substrate through said selected porous regions comprises use of capillary action.

32. The method of claim 9, where said withdrawing said excess reagent from said substrate through said selected porous regions comprises use of gravity.

33. The method of claim 9, wherein said providing said substrate comprises:
(a) providing a base; and
(b) joining said base to said substrate to define an enclosure between said base and said substrate.

34. The method of claim 33, wherein said providing said substrate further comprises providing a gasket configured to sealingly engage said substrate and said base.

35. The method of claim 9, wherein said contacting said isolated porous regions with said reagents is carried out with a liquid dispenser head.

36. The method of claim 33, wherein said providing said substrate further comprises providing support element for substrate, said support element including a plurality of holes, said substrate and said support element configured to align said plurality of isolated porous regions of said substrate with said plurality of holes of said support element.

37. The method of claim 9, wherein said isolated porous regions are present on said substrate at a density of between about 1 porous region per square centimeter and about 10 porous regions per square centimeter.

38. The method of claim 9, wherein said isolated porous regions are present on said substrate at a density of between about 10 porous regions per square centimeter and about 100 porous regions per square centimeter.

39. The method of claim 9, wherein said isolated porous regions are present on said substrate at a density of between about 100 porous regions per square centimeter and about 10000 porous regions per square centimeter.

40. The method of claim 9, wherein said isolated porous regions are present on said substrate at a density of between about 1000 porous regions per square centimeter and about 100000 porous regions per square centimeter.

41. The method of claim 9, wherein said isolated porous regions are present on said substrate at a density of between about 10000 porous regions per square centimeter and about 100000 porous regions per square centimeter.

42. The method of claim 9, wherein said isolated porous regions are present on said substrate at a density of between about 100000 porous regions per square centimeter and about 1000000 porous regions per square centimeter.

43. The method of claim 12, wherein said nucleic acid comprises of between about 2 nucleic acid bases and about 100 nucleic acid bases.

44. The method of claim 12, wherein said nucleic acid comprises of between about 100 nucleic acid bases and about 1000 nucleic acid bases.

45. The method of claim 12, wherein said nucleic acid comprises of between about 1000 nucleic acid bases and about 10000 nucleic acid bases.

46. The method of claim 12, wherein said nucleic acid comprises of between about 10000 nucleic acid bases and about 100000 nucleic acid bases.

47. The method of claim 8, wherein each of said porous regions includes a plurality of pores with an average pore size of between about 0.1 millimeter in diameter and about 1 millimeter in diameter.

48. The method of claim 8, wherein each of said porous regions includes a plurality of pores with an average pore size of between about 0.1 millimeter in diameter and about 10 micron in diameter.

49. The method of claim 8, wherein each of said porous regions includes a plurality of pores with an average pore size of between about 1 micron in diameter and about 10 micron in diameter.

50. The method of claim 8, wherein each of said porous regions includes a plurality of pores with an average pore size of between about 1 micron in diameter and about 100 nanometers in diameter.

51. The method of claim 8, wherein each of said porous regions includes a plurality of pores with an average pore size of between about 100 nanometer in diameter and about 1 nanometer in diameter.

52. The method of claim 8, wherein said contacting said porous regions with said reagent comprises applying a liquid reagent having a volume of between about 1 milliliter and about 100 microliters.

53. The method of claim 8, wherein said contacting said porous regions with said reagent comprises applying a liquid reagent having a volume of between about 100 microliters and about 1 microliter.

54. The method of claim 8, wherein said contacting said porous regions with said reagent comprises applying a liquid reagent having a volume of between about 1 microliter and 100 nanoliters.

55. The method of claim 8, wherein said contacting said porous regions with said reagent comprises applying a liquid reagent having a volume of between about 100 nanoliters and 1 nanoliter.

56. The method of claim 8, wherein said contacting said porous regions with said reagent comprises applying a liquid reagent having a volume of between about 1 nanoliter and about 1 picoliter.

57. The method of claim 8, wherein said contacting said porous regions with said reagent comprises applying a liquid reagent having a volume of between about 1 picoliter and about 1 femtoliter.

58. The method of claim 8, further comprising cleaving said nucleic acids from said substrate.

59. The method of claim 49, further comprising carrying out a polymerase chain reaction using said nucleic acids cleaved from said substrate to make copies of said nucleic acids.

60. The method of claim 49, further comprising coupling said nucleic acids together.

61. The method of claim 49, further comprising inserting said cleaved nucleic acids into a DNA molecule to provide a mutation therein.

62. The method of claim 33, further comprising coupling said enclosure to a vacuum source.
